(19) United States
(12) Patent Application Publication
(10) Pub. No.: US 2005/0159713 A1
(43) Pub. Date: Jul. 21, 2005

(54) Portable and self-contained lavage apparatus

(76) Inventor: Robert McPherson, Amanda Park, WA (US)

Correspondence Address: Charles J. Rupnick, PO Box 46752, Seattle, WA, 98146, US

(21) Appl. No.: 10/758,988

(22) Filed: Jan. 16, 2004

(51) Int. Cl. A61M 31/00

(52) U.S. Cl. 604/279

(57) ABSTRACT

A lavage apparatus is formed of a hand-operable pump embodied as a single bulb having a thin-walled outer shell of a resiliently deformable plastic material with an interior surface forming a water solution cavity or reservoir; an integral spray wand formed of an elongated thin-walled cylindrical tube of substantially rigid plastic material with an integral coupler formed at one end that communicates with the water solution cavity of the pump, and having a free end distal from the coupler; and a single aperture thin-walled cylindrical tube shaped spray nozzle oriented at a right angle to the elongated tube of the spray wand to jet a single stream of liquid crosswise to the elongated tube of the spray wand.

### FIELD OF THE INVENTION

The present invention relates to hygienic apparatuses and methods, and in particular to hand-held portable lavage apparatuses.

### BACKGROUND OF THE INVENTION

An age-old concept of personal hygiene of your intimate areas with water is still common practice today by using a bidet. Personal rinsing was developed by physicians and nurses for protection of personal health. Rubbing with paper has been found both unclean and archaic; it may also be very irritating to delicate tissues and spreads bacteria around the rectal and vaginal areas. The resulting contamination can cause discomfort and lead to vaginal colonization.

The problem is more than one of aesthetics and discomfort. Using toilet paper has been found to be a major cause of bladder and urinary tract infections. For example, the vast majority of urinary infections may be caused by E. coli and other intestinal pathogens. Sexual activity may exacerbate the problem by further spreading of bacteria in the perineal area. The annual morbidity from urinary infections and resultant costs to patients and society is staggering. Thorough cleansing with water dramatically lessens the toll of urinary infections. Personal hygiene of your intimate areas with water by using a bidet is effective at reducing or eliminating urinary tract infections. Daily washing with a bidet has been shown to aid and relieve some of the most common-ailments that may occur. For example, hemorrhoids, urinary tract infections, diarrhea, feminine discomforts may be relieved by washing with a bidet.

Hemorrhoids are a serious health problem that is irritated by toilet paper, but relieved by water cleansing. About 75% of all Americans suffer from hemorrhoids or will at some time in their lives. Besides itching and bleeding, hemorrhoids make it difficult to clean the rectal area which leads to even more rubbing and irritation. Sitz baths and water soaks have long been used to sooth and help heat hemorrhoids. Bidets are often recommended as a way to cleanse without the mechanical irritation of toilet paper. Washing with a bidet cleans the entire perineum, including the tissue around hemorrhoids, without manual contact or rubbing of any kind. Daily washing with a bidet provides relief from warm soothing water spray and relieves aggravation of the affected and surrounding areas by reducing irritating itching, swelling, and even bleeding.

Washing with a bidet also provides gentle, non-irritating care for many other health problems including rashes, fissures and postnatal care for new mothers. Feminine deodorants, douches, wipes and other commercial feminine hygiene items may be potentially harmful due to chemicals they may have. Daily washing with a bidet using pure natural water to cleanse has been shown to relieve minor feminine discomforts during menstruation and dramatically provide relief from other small common infections and discomforts.

Maintaining personal cleanliness is extremely difficult for many arthritic and handicapped people; continuous daily washing with a bidet can provide benefits that make life measurably more comfortable and pleasant while allowing the person to use the bathroom independently.

In addition to the personal health benefits, washing with a bidet can help decrease or eliminate widespread fecal contamination in homes with young children, disabled or elderly persons. Because washing with a bidet cleans without touching, fecal contamination of hands, skin, clothing and bathroom fixtures can be reduced or eliminated.

The use of a bidet is also believed to teach children good hygiene essentials.

However, the cost and space requirements of traditional bidets make them unobtainable to most ordinary home owners. Therefore, bidets have been developed that are retrofitable on an original toilet. Such retrofitable bidets are externally attachable to transform an original toilet into a bidet without expensive and complicated plumbing alterations. These retrofitable bidets are touted as an economical way to add freshness and health to a daily hygienic routine. Cold and hot/cold water retrofitable bidet systems have been developed that fit both standard toilets as well as one-piece toilets. Such retrofitable bidet systems attach to the toilet bowl and water tank supply and are composed, for example, of a compact spray arm mounted under the rim of the toilet bowl. A handle positions a spray arm to deliver a shower of fresh tap water to cleanse. Release of the handle causes the spray arm to automatically shut off and causes the spray arm to return to its resting position under the rim. Such retrofitable bidet systems are constructed from a plurality of very specialized materials, including for example, stainless steel, rubbers, reinforced fiber compounds, high tensile brass, spun aluminum, ABS, nylon, high-pressure polypropylene and crystal clear polycarbonate. Each material plays a special role in the performance, reliability and aesthetic appearance of the bidet device.

Some bidet users have become so reliant on the health and comfort of washing with a bidet that they don't like to travel because hotels do not ordinarily supply a bidet. These dedicated users have created a demand for a portable bidet. There are currently such portable bidets commercially available so that users can enjoy water cleansing away from home. Many of these portable bidet devices must be fitted to a toilet bowl and connected to an external source of fresh water before they are usable. Most of these devices controllably dispense a contained water solution under pressure; some of these portable bidet devices must even be plugged into an electrical outlet to operate.

Other portable bidets are hand-held devices but are typically complex and include a compact water reservoir coupled to an extendable spray wand that emits several streams or jets of water. One such hand-held portable bidet device holds warm or cold water and provides a pulse function operated by two AA size batteries. The water jet produced by this battery operated device is reputed to contain air bubbles to give it a required volume. Furthermore, the water jet is reputed to sway very slightly from side to side as it washes to ensure complete cleansing.

U.S. Pat. No. 5,864,895, HANDY BODY WASHER, issued to Ota, et al. on Feb. 2, 1999, the entirety of which is incorporated herein by reference, illustrates another battery-operated hand-held portable bidet device includes a main body, a tank slidably attached to the main body for storing washing water, a retractable nozzle having a plurality of nozzle apertures for dispensing the washing water from the tank, and a battery-operated pump for jetting out the washing water through the nozzle apertures. The tank is a bottomed cylindrical body having an open top end and a bottom end, the bottom end having a water supply opening. A water supply cover is pivotally supported on the bottom end of the tank for opening and closing the water supply opening, and a gutter is formed in an inner surface of the water supply cover for introducing the washing water flowing downward into the water supply opening while the water supply cover is open. Since the water supply opening is arranged on the bottom of the tank, a large part of the main body can be received in the tank during carriage, thereby reducing the whole volume of the device for portability during carriage. While the water supply cover is open to supply water to the tank, the gutter arranged on the inner surface of the water supply cover turns the flow of water toward the opening to fit the device in a small space between a faucet and basin for filling. The main body includes a waterproof switch, a battery cell, a motor that uses the battery cell as a power source, and the washing water pump driven by the motor. A press of the switch actuates the motor to drive the pump which pumps out the washing water stored in the tank and causes the washing water to be fed to the nozzle through a feed pipe and to be jetted out of the jet nozzles (or nozzle apertures) of the nozzle. The battery-operated hand-held portable bidet device of Ota, et al. is used by holding the main body and the tank with a hand and pressing the switch with the thumb. When the user presses the switch while facing the jet nozzles of the nozzle toward the posterior parts or the external genitals of the body, the washing water showers the posterior parts or the external genitals of the body. In the course of washing, the washing water in the tank gradually decreases. An air valve disposed on the bottom of the tank allows the air flow between the sealed space in the tank and the exterior. When the main body is slid into the tank for reducing the whole volume of the device during carriage, the air valve releases the air remaining in the tank to the atmosphere so that the main-body can be slid into the tank.

Thus, currently known hand-held portable bidet devices are extremely complex, often requiring an electrical outlet or other power supply for operation. As a result of this complexity, currently known hand-held portable bidet devices are expensive and their hygienic and other beneficial effects are limited to users of financial means.

### SUMMARY OF THE INVENTION

The present invention overcomes obvious limitations of the prior art by providing a lavage apparatus that is formed of a hand-operable pump embodied as a single bulb having a thin-walled outer shell of a resiliently deformable plastic material with an interior surface forming a water solution cavity or reservoir; an integral spray wand formed of a substantially straight and elongated thin-walled cylindrical tube of substantially rigid plastic material with an integral coupler formed at one end that communicates with the water solution cavity of the pump, and having a free end distal from the coupler; and a single aperture thin-walled cylindrical tube shaped spray nozzle oriented at a right angle to the elongated tube of the spray wand to jet a single stream of liquid crosswise to the elongated tube of the spray wand.

According to another aspect of the invention, the single aperture spray nozzle is formed of a substantially cylindrical interior flow channel communicating with a single interior flow channel of the elongated thin-walled cylindrical tube of the spray wand.

According to another aspect of the invention, the single interior flow channel of the spray nozzle measures about one quarter inch in diameter.

According to another aspect of the invention, the coupler is formed of a plug of substantially larger diameter than the elongated thin-walled cylindrical tube portion of the spray wand, the plug having an interior flow channel communicating between the water solution cavity of the pump and the elongated thin-walled cylindrical tube portion of the spray wand.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a pictorial perspective view of the lavage apparatus of the invention embodied in hand-held and self-contained portable bidet for hygienic cleansing of intimate body areas with water or a water-based solution;

FIG. 2 is a cross-sectional view lengthwise through the portable bidet of the invention that shows the bulb as a bulbous shape in its relaxed state and having its entire interior being formed of a water solution cavity or reservoir for holding water or a water solution to be used for hygienic cleansing;

FIG. 3 illustrates one alternative embodiment of the spray wand portion of the portable bidet of the invention having a spray tip and single spray nozzle formed in a separate unit from the remainder of the spray wand; and

FIG. 4 illustrates yet another alternative embodiment of the separate spray wand portion of the portable bidet of the invention.

### DETAILED DESCRIPTION OF PREFERRED EMBODIMENT

In the Figures, like numerals indicate like elements.

The present invention is a lavage apparatus embodied in hand-held portable bidet formed of a manually operated pump having a single internal water solution cavity of fixed dimensions and volume and a single opening for filling and emptying the water solution cavity, a substantially straight and elongated tubular spray wand of fixed length having a first end fixed to the opening in the pump and a second free end distal from the pump opening, and a spray tip coupled to the free end of the spray wand and rotated relative thereto with a single aperture spray nozzle oriented crosswise to the length of the spray wand for jetting a portion of the water solution therefrom when the manually operated pump is squeezed.

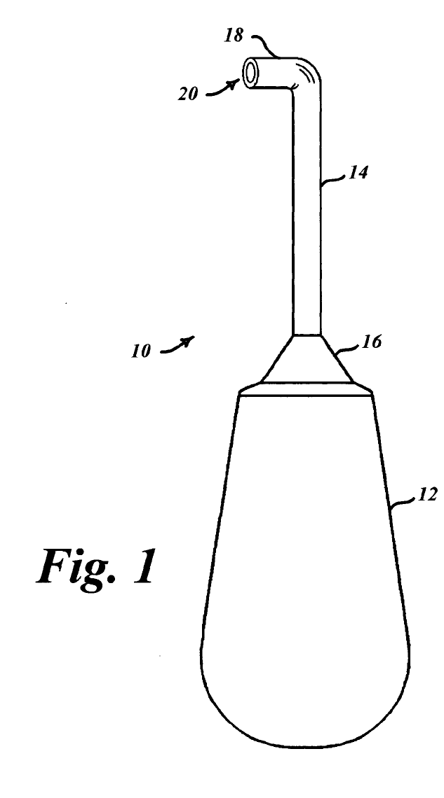

FIG. 1 is a pictorial perspective view of the lavage apparatus of the invention embodied in hand-held and self-contained portable bidet 10 for hygienic cleansing of intimate body areas with water or a water-based solution. The portable bidet 10 has a one-piece bulb 12 coupled to a completely separate substantially straight and elongated spray wand 14. An integral coupler 16 joins a near end of the spray wand 14 to the bulb 12. The substantially straight and elongated spray wand 14 has at its far end a spray tip 18 that is rotated relative to the main body of the spray wand 14 and provided with a single spray nozzle 20 oriented crosswise to the spray wand 14.

In order to be both portable and self-contained while remaining easy and comfortable to use, the bulb 12 is sized to fit in the user's hand for comfortable hand-squeezing of the manually operable pump feature, while the spray wand 14 is sized to reach intimate body areas while the user is in a seated position. The hand-held and self-contained portable bidet 10 of the invention is thus presented as having an overall length in the range of about 10 to 12 inches, but may be slightly shorter or longer without materially effecting the practice of the invention. For example, according to one embodiment of the invention, the overall length, including the spray tip 18 containing the spray nozzle 20, is about 11 1/2 inches.

The portable bidet 10 is used while seated on a commode. Fresh water or a water-based solution is introduced into the bulb 12, either directly through an opening exposed by de-coupling the spray wand 14, or indirectly by siphoning through the spray wand 14. With the liquid filled bulb 12 in held in one hand with the spray wand 14 in a generally downward pointing orientation and the spray nozzle 20 in a generally upward pointing orientation, the user introduces the spray nozzle 20 to the intimate body area. Squeezing the bulb 12 operates to pump the liquid from the bulb 12 into and through the elongated spray wand 14 and spray tip 18, jetting the liquid in a large stream out through the spray nozzle 20. The force of the jetted stream is controlled by the pressure exerted on the bulb 12 by the user. A thorough hygienic cleansing is accomplished with absolutely no irritation.

FIG. 2 is a cross-sectional view lengthwise through the portable bidet 10 of the invention that shows the bulb 12 as a bulbous shape in its relaxed state and having its entire interior being formed of a water solution cavity or reservoir 22 for holding water or a water-based solution to be used for hygienic cleansing. The bulb 12 is, by example and without limitation, of a type of slightly elongated bulb that is part of a conventional basting syringe of a type used for basting roasts while cooking. Accordingly, the bulb 12 is of permanently fixed dimensions sized to conveniently fit in the user's hand, and the water solution cavity or reservoir 22 is sized to hold an amount of liquid convenient for basting a roast fowl. By example and without limitation, the bulb 12 measures about 3 to 4 inches in length by about 2 to 3 inches in diameter, and the reservoir 22 is sized to hold about ½ cup to a full cup, or 4 to 8 ounces of warm or cool water, but may be slightly smaller or larger without materially effecting the practice of the invention. For example, according to one embodiment of the invention, the bulb 12 measures about 3 1/2 inches in length by about 2 1/2 inches in diameter, and the reservoir 22 is sized to hold about ¾ cup or 6 ounces of warm or cool water. When the bulb 12 is of a type used in cooking, it is formed of material that is resistant to chemicals and harsh cleaning agents so that it may be readily cleaned.

The bulb 12 is formed of a thin-walled exterior shell 24 having an interior surface 25 whereof the water solution reservoir 22 is formed. The bulb 12 design is limited to the exterior shell 24 which completely contains the reservoir 22, a resiliently expandable mouth 26 communicating with the shell exterior, and a short resiliently expandable throat 28 communicating between the reservoir 22 and the mouth 26. This simple and open design permits the bulb 12 to be easily and thoroughly cleaned inside and out and thoroughly rinsed of any harsh chemicals or cleaning agents so that the user's health and comfort are never compromised.

Furthermore, the bulb 12 is formed of a resiliently deformable material such as plastic, rubber or another suitably resiliently deformable material so that the shell 24 is readily compressed to deflate the reservoir 22 which thereby forces the liquid therefrom through the elongated spray wand 14 and spray tip 18 for jetting the liquid in a large stream from the spray nozzle 20. The resiliently deformable material of the bulb 12 permits the shell 24 to be resiliently compressed when the reservoir 22 is empty or only partially filled, whereby a vacuum is formed in the reservoir for siphoning fresh water or water-based solution thereinto through the spray wand 14.

The expandable mouth 26 and the short expandable throat 28 communicating between the reservoir 22 and the mouth 26 are sized to accept thereinto the coupler 16 that is integral with a near end 30 of the spray wand. 14. The coupler 16 is a hollow tube shape having a first larger diameter plug 32 that is sized slightly larger in diameter than the expandable mouth 26 and throat-28 portions of the bulb 12 such that, when forced thereinto, the plug 32 expands the mouth 26 and throat 28 portions and couples snugly therewith. The plug 32, and with it the remainder of the spray wand 14, thus communicates directly with the bulb reservoir 22 without need for an inner siphon or “dip” tube of the type typical of spray bottles and atomizers that are used for reaching the liquid at the bottom of the reservoir, as illustrated for example in U.S. Pat. No. 4,503,996, LIQUID ATOMIZER HAVING A DOUBLE-ACTING PUMP, issued to Sorm, et. al. on. Mar. 12, 1985, which is incorporated herein by reference. The plug 32, as well as the remainder of the spray wand 14, is formed of a substantially rigid and inflexible material, such as a hard plastic or metal, having substantially smooth interior and exterior surfaces. The rigidity and smoothness of the material permits the plug 32 to be forced into the slightly smaller mouth 26 and throat 28 of the bulb 12 and to form a substantially water-tight seal therewith, whereby water or other liquid solution in the reservoir 22 is prevented from leaking from the bulb mouth 26. Additionally, the exterior of the plug 32 is optionally formed with a slightly conical “bayonet” shape (indicated at 32) that provides a lead-in for gradually expanding the bulb mouth 26 and throat 28 during installation of the spray wand 14. Another optional feature of the plug 32 is a relief 34 formed away from its interior flow channel 36. The optional relief 34 permits a cooperating portion of the bulb throat 28 to flow thereinto for capturing the plug 32 and fixing the spray wand 14 relative to the bulb 12. An optional lip 38 formed on an exterior plug surface operates as a stop for fixing the plug 32 against the mouth 26 of the bulb 12. The length of the plug bayonet portion (indicated at 32) is such that the plug 32 extends about the same or slightly more (shown) than the length of the throat 28 so that the interior flow channel 36 of the plug 32 communicates with the bulb reservoir 22 without entering appreciably thereinto.

The interior flow channel 36 of the coupler 16 includes an interface channel 40 enclosed in a fluted or conical (shown) casement 42 between an enlarged portion 44 of the plug's interior flow channel 36 and a much reduced cylindrical interior flow channel 46 of a substantially straight, rigid and inflexible elongated thin-walled tube 48 that is integrally formed with the casement 42 portion of the coupler 16. The cylindrical interior flow channel 46 of the elongated tube 48 is, by example and without limitation, of substantially constant cylindrical cross-section on the order of ¼ inch in diameter, but may be slightly smaller or larger without materially effecting the practice of the invention. The elongated tube 48 of the spray wand 14 extends from the casement 42 portion of the coupler 16 for a permanently fixed and unalterable length of about 6 to 8 inches to the spray tip 18 at its far end 50 distal from the bulb 12. However, the tube 48 of the spray wand 14 may be longer or even slightly shorter without materially effecting the practice of the invention. By example and without limitation, according to one embodiment of the invention, the tube 48 of the spray wand 14 is about 6 1/2 inches long.

The cylindrical interior flow channel 46 of the elongated tube 48 is continuous through the spray tip 18 that is, according to one embodiment of the invention, a continuation of the substantially straight, rigid and inflexible thin-walled tube 48 that forms the length of the spray wand 14. Thus, according to one embodiment of the invention, the single spray nozzle 20 is formed as a substantially rigid and inflexible thin-walled tube having an cylindrical interior flow channel 52 that is a continuation of the cylindrical interior flow channel 46 of the elongated tube 48. That is to say, according to one embodiment of the invention, the flow channel 52 that forms the single spray nozzle 20 is formed as a cylinder having an inner diameter on the order of about 1/4 inch, but which may be slightly smaller or larger without materially effecting the practice of the invention. The spray tip 18 is rotated at a right angle relative to the substantially straight elongated tube 48 portion of the spray wand 14 so that the single spray nozzle 20 is oriented substantially crosswise to the remainder of the spray wand 14, i.e., crosswise to the substantially straight elongated tube 48. However, the angle of rotation of the spray tip 18 relative to the elongated tube 48 can vary considerably from the right angle depicted without materially effecting the practice of the invention. For example, the crosswise orientation of the spray tip 18 relative to the elongated tube 48 can vary as much as 15 degrees or even 30 degrees from the right angle depicted without materially effecting the practice of the invention. According to one embodiment of the invention, the spray tip 18 extends for a permanently fixed and unalterable length about 1 inch from the elongated tube 48 portion of the spray wand 14, but may extend any appropriate length that effectively serves to direct a large substantially coherent stream of liquid jetted from the portable bidet 10 along a path oriented for a short distance substantially crosswise to the substantially straight elongated tube 48. The single flow channel 52 that forms the single spray nozzle 20 thus provides a single washing mode formed of one large and substantially coherent stream of water that is generated by squeezing the bulb 12.

According to the invention, warm or cool water or another water-based hygienic cleansing solution is introduced into the reservoir 22 of the bulb 12. As desired or as the water supply design permits, liquid can be introduced into the reservoir 22 directly by removing the spray wand 14 from the bulb 12 and pouring the liquid into the reservoir through the open mouth 26 and throat 28. Alternatively, liquid can be introduced into the reservoir 22 indirectly by leaving the spray wand 14 coupled to the bulb and siphoning liquid through the spray nozzle 20 into the consecutive interconnected interior flow channels 52, 46 and 36 and thereafter into the reservoir 22. Such siphoning of the liquid can be accomplished by deformably compressing the bulb shell 24, placing the spray nozzle 20 in a passive source liquid, such as a bowl or sink, or an active source, such as faucet having a flowing stream of water. The compressed bulb shell 24 is released whereupon the resiliently deformable material returns to its original relaxed bulbous shape which thereby creates a vacuum in the reservoir 22. The resultant vacuum causes a siphon that draws the liquid into the bulb reservoir 22. When the liquid is drawn from an active source, the pressure of the flowing stream is used to force the liquid into the bulb reservoir 22. Compression of the bulb shell 24 and the resultant siphon from subsequent decompression can be used in combination with the pressure of the flowing stream to fill the reservoir 22 when the liquid is drawn from an active source.

Subsequent compression of the bulb shell 24 forces the liquid from the bulb reservoir 22, through the consecutive interconnected interior flow channels 36, 46 and 52 and thereafter out through the spray nozzle 20 in a single substantially coherent stream of cleansing liquid that is large enough to effectively “sweep clean” the user's entire rectal or vaginal region, even if the user's aim is less than optimal, by merely waving the spray wand 14 in over the region. The generous proportions of the reservoir 22 guarantee that the user can squirt two or even three or more consecutive cleansing streams without stopping to refill.

The portable bidet 10 of the present invention thus provides a simple lavage pump that is manually operated by hand-squeezing of the resiliently deformable bulb shell 24 and delivers a single large and substantially coherent stream of cleansing liquid from the simple tubular spray nozzle 20 in a single washing mode. The portable bidet 10 of the present invention thereby overcomes complexity and expense limitations of the prior art by eliminating the complex multi-nozzle spray heads delivering multiple streams of water by means electrically powered pumps.

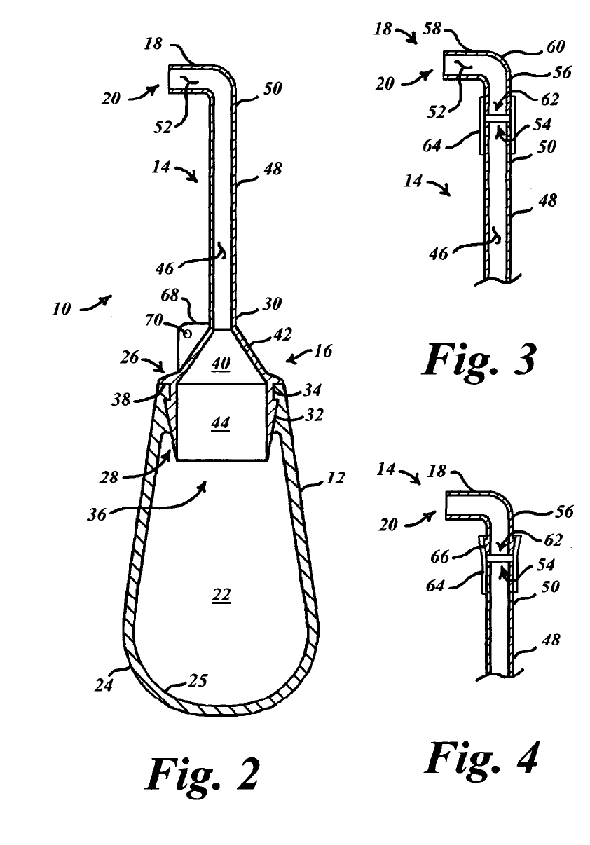

FIG. 3 illustrates one alternative embodiment of the spray wand 14 having the spray tip 18 with its single spray nozzle 20 formed in a separate unit from the elongated tube 48 and the remainder of the spray wand 14. Accordingly, the main spray wand 14 includes the elongated tube 48 embodied as described herein, except the interior flow channel 46 culminates in an opening or mouth 54 at the tube's far end 50. The spray tip 18 includes the interior flow channel 52 that culminates in the single spray nozzle 20 embodied as described herein. However, the spray tip 18 is embodied as a single elbow joint formed of substantially identical near and far tube sections 56, 58 interconnected by a curved portion 60 of the of the interior flow channel 52 wherein the near tube section 56 culminates in an opening or mouth 62 that matches the mouth 54 at the far end 50 of the elongated tube 48. A sleeve 64 of a resiliently expandable tubing material, such as plastic, rubber, vinyl, or another suitably resiliently expandable material, that is sized to snuggly fit over both the elongated tube 48 and the near tube section 56 of the spray tip 18 and form a substantially water-tight seal therebetween, whereby the respective interior flow channels 46 and 52 of the elongated tube 48 and the spray tip 18 form a single continuous flow channel with each other and the plug flow channel 36, as described herein. It is known that tubing materials of which the sleeve 64 may be formed are often supplied in tightly coiled rolls. Residual curvature in the sleeve 64 may cause slight variations in the alignment of the spray tip 18 and spray nozzle 20 relative to the elongated tube 48 without materially effecting the practice of the invention.

FIG. 4 illustrates yet another alternative embodiment of the spray wand 14 having the spray tip 18 with its single spray nozzle 20 formed in a separate unit from the elongated tube 48 and the remainder of the spray wand 14. Accordingly, the opening or mouth 62 in the near tube section 56 is formed with a slightly conical “bayonet” shape 66 for more definitely coupling with the resiliently expandable sleeve 64 interconnecting the near tube section 56 with the far end 50 of the elongated tube 48 in a substantially water-tight seal.

According to yet other alternative embodiments of the hand-held and self-contained portable bidet 10 of the invention, means are provided for conveniently hanging or carrying the portable bidet 10. By example and without limitation, the hanging or carrying means is embodied, as illustrated in FIG. 2, as a small tab 68 integrally formed with a portion of the spray wand 14 such as the conical or fluted casement 42 portion of the plug 32. A connector means 70, illustrated as a hole, is formed through the thickness of the tab 68. Thereafter, the hole of connector means 70 is used to hang the portable bidet 10 on a conveniently located hook or other small protuberance. Alternatively, the hole of connector means 70 is used to attach a lanyard or thong for easy carrying of the portable bidet 10.

While the preferred embodiment of the invention has been illustrated and described, it will be appreciated that various changes can be made therein without departing from the spirit and scope of the invention. For example, the coupler 16 joining the spray wand 14 to the bulb 12 is optionally formed with the plug 32 having generously proportioned external screw threads in place of the bayonet shape illustrated, while the throat 28 of the bulb 12 is formed with matching internal screw threads, whereby the spray wand 14 is optionally coupled to the bulb 12 by threading. The threading in turn draws the lip 38 of the plug 32 against the mouth 26 of the bulb 12 to form the substantially water-tight seal therebetween. Other changes can also be made therein without departing from the spirit and scope of the invention.

1. A lavage apparatus, comprising:
a manually operable pump having a single internal water solution cavity of fixed dimensions and volume and a single opening for filling and emptying the water solution cavity;
an elongated tubular spray wand of fixed length having a first end fixed to the opening in the pump and a second free end distal from the pump opening, and
a spray tip coupled to the free end of the spray wand and angularly rotated relative thereto, the spray tip having a single aperture spray nozzle oriented crosswise to the length of the spray wand for jetting a portion of water solution from the internal water solution cavity when the manually operated pump is operated.

2. The lavage apparatus of claim 1 wherein the manually operable pump further comprises an outer shell having the single opening formed therein and containing the single water solution cavity.

3. The ravage apparatus of claim 2 wherein the outer shell of the manually operable pump further comprises a resiliently deformable material.

4. The lavage apparatus of claim 1 wherein the first end of the spray wand further comprises an enlarged plug sized to fit into the single opening in the pump and form a substantially water-tight seal therewith.

5. The lavage apparatus of claim 4 wherein the enlarged plug further comprises an interior flow channel extending therethrough and communicating between the internal water solution cavity of the pump and an elongated tubular portion of the spray wand.

6. The lavage apparatus of claim 1 wherein the spray tip further comprises a continuous tubular interior flow channel extending therethrough and forming the single aperture spray nozzle.

7. A lavage apparatus, comprising:
a resiliently deformable bulb containing a water solution reservoir;
a substantially rigid elongated spray wand having an interior flow channel extending therethrough and communicating with the water solution reservoir; and
a tubular spray nozzle extending at an angle from a far end of the elongated spray wand distal from the bulb and having an interior flow channel extending therethrough and communicating with the interior flow channel of the spray wand.

8. The lavage apparatus of claim 7 wherein the resiliently deformable bulb further comprises a shell formed of resiliently deformable and having an interior surface whereof the water solution reservoir is formed.

9. The lavage apparatus of claim 7 wherein the bulb further comprises a resiliently expandable mouth communicating between the water solution reservoir and an exterior surface of the bulb.

10. The lavage apparatus of claim 9 wherein the spray wand is removably coupled to the bulb in a substantially water-tight joint with the mouth.

11. The lavage apparatus of claim 7 wherein the spray nozzle further comprises thin-walled tube having a single substantially cylindrical interior flow channel extending therethrough.

12. The lavage apparatus of claim 11 wherein the single substantially cylindrical interior flow channel extending through the thin-walled tube of the spray nozzle further comprises a single substantially cylindrical interior flow channel having an inside diameter of about one quarter inch.

13. The lavage apparatus of claim 7 wherein the angle at which the spray nozzle extends an from a far end of the elongated spray wand further comprises a right angle.

14. The lavage apparatus of claim 7 wherein the elongated spray wand measures about six to eight inches in length.

15. A lavage apparatus, comprising:
a hand-operable pump formed of a single bulb having an outer shell of resiliently deformable material with an interior surface forming a water solution cavity;
an integral spray wand formed of an elongated thin-walled cylindrical tube of substantially rigid material with an integral coupler formed at one end and communicating with the water solution cavity of the pump, and having a free end distal from the coupler; and
a single aperture spray nozzle structured to direct a single stream of liquid crosswise to the elongated tube of the spray wand.

16. The lavage apparatus of claim 15 wherein the single aperture spray nozzle is oriented crosswise to the elongated tube of the spray wand.

17. The lavage apparatus of claim 16 wherein the single aperture spray nozzle is oriented at a right angle to the elongated tube of the spray wand.

18. The lavage apparatus of claim 15 wherein the single aperture spray nozzle further comprises a single substantially cylindrical thin-walled tube portion having a substantially cylindrical interior flow channel communicating with a single interior flow channel of the elongated thin-walled cylindrical tube of the spray wand.

19. The lavage apparatus of claim 18 wherein the single interior flow channel of the spray nozzle further comprises a diameter that measures about one quarter inch.

20. The lavage apparatus of claim 15 wherein the coupler further comprises:
a plug of substantially larger diameter than the elongated thin-walled cylindrical tube portion of the spray wand, and
an interior flow channel communicating between the water solution cavity of the pump and the elongated thin-walled cylindrical tube portion of the spray wand.
